(19) United States
(12) Patent Application Publication
(10) Pub. No.: US 2014/0066741 A1
(43) Pub. Date: Mar. 6, 2014

(54) Disposable ECG Leadwire Connector

(71) Applicants: James Russel Peterson, Milwaukee, WI (US); Michael P. McLeod, Milwaukee, WI (US); Brian Behl, Milwaukee, WI (US); Jason Christian, Milwaukee, WI (US)

(72) Inventors: James Russel Peterson, Milwaukee, WI (US); Michael P. McLeod, Milwaukee, WI (US); Brian Behl, Milwaukee, WI (US); Jason Christian, Milwaukee, WI (US)

(73) Assignee: GENERAL ELECTRIC COMPANY, Schenectady, NY (US)

(21) Appl. No.: 13/598,022

(22) Filed: Aug. 29, 2012

(51) Int. Cl. A61B 5/0408

(52) U.S. Cl. 600/393

(57) ABSTRACT

The ECG leadwire connector is designed in a way that allows low cost high volume manufacturing of an ECG leadwire assembly using standard continuous web converting processes and materials. The integrated ECG leadwire connector is assembled using the same printing and laminating processes that are used to construct the rest of the leadwire assembly. Patient safety requirements are met by the physical dimensions of the connector electrical contacts and by the thickness of a dielectric isolation feature which prevents inadvertent contact with potentially hazardous potentials, while still allowing the connector pins to make contact with the leadwire conductors. The connector also incorporates and RFID tag that allows multiple leadwire configurations to be accommodated and identified and authenticated during use.

### FIELD

The present application is directed to the field of ECG leadwire connectors. More specifically, the present application is directed to the field of disposable ECG leadwire applications and connectors.

### BACKGROUND

Previous disposable leadwire connectors did not adequately address the patient safety or radio frequency identification (RFID) features incorporated into the design of the present application. Accordingly, current leadwire connectors cannot be made to be disposable and compatible with the manufacturing processes used to construct leadwires on flexible printed substrate materials.

Section 4.2.1 of the Association for the Advancement of Medical Instrumentation (AAMI) EC53 standard refers to the DIN 42-802 standard for ECG leadwire connector requirements to mitigate the potential hazard of a patient connected leadwire inadvertently making electrical contact with the power mains or other hazardous voltage sources. The DIN 42-802 standard specifies that a standard test probe such as a finger not be able to make electrical contact with the ECG leadwire connector pins (or sockets) when applied with a force of 30 N (6.744 lbs.). Section 8.5.2.3 of the AAMI ES60601-1 2005 C1 2009 has similar requirements except the test probe force is reduced to 10 N (2.248 lbs.). In addition the ES60601-1 standard requires 1.0 mm (0.039 in.) creepage distance, 0.5 mm (0.0196 in.) of air clearance, and a dielectric strength of 1,500 Vac for at least 1 minute, protection from mains voltage.

Defibrillation patient safety requirements of AAMI EC11:1991/(R)2001 (section 3.2.14.2.2) dictate that the reduction in energy delivered to a patient being defibrillated be less than 10% of the total energy delivered by the defibrillator while an electrocardiograph and its associated leadwires are attached to the patient in order to maintain the efficacy of defibrillation. The defibrillator generates voltages of up to 5000V peak, therefore, in order to prevent “arcing” of defib energy shunting around the patient, the leadwires and the connector must maintain adequate electrical isolation (i.e., withstand 5000V peak). In order to guarantee this level of isolation a 8.0 mm (0.314 in.) creepage distance, 4.0 mm (0.157 in.) of air clearance must be maintained in order to support a dielectric breakdown strength of 5,000 volts between the exposed conductive surfaces of the connector. These dimensional requirements are based upon the assumptions of a pollution degree 2 level, using materials with a comparative tracking index (CTI) greater than 175 for the substrate, at an altitude less than 2000 meters above sea level. The non-exposed conductor areas of the connector/leadwire must also be constructed to support the 5,000 volt withstand level by incorporating appropriate conductor spacing's based upon the specific dielectric materials inbetween them.

### SUMMARY

In one aspect of the present application, an electrocardiograph (ECG) leadwire connector includes a plurality of electrode contacts, a plurality of connector pins corresponding to, and electrically coupled with each of the plurality of electrode contacts, wherein the plurality of connector pins and the plurality of electrode contacts are covered by a flexible dielectric substrate, the flexible dielectric substrate forming the ECG leadwire connecter, a stiffener area formed over the plurality of electrode contacts and affixed to the flexible dielectric material, a plurality of apertures on a first side of the stiffener area and the flexible dielectric substrate wherein the plurality of apertures expose a first side of each of the plurality of electrode contacts, and a plurality of alignment slots configured through the stiffener area and the flexible dielectric material, wherein the plurality of alignments slots define a separation between each of the plurality of electrode contacts.

In another aspect of the present application, an electrocardiograph (ECG) leadwire system includes a ECG leadwire connector fashioned from a flexible dielectric substrate having a plurality of electrode contacts and a plurality of connector pins corresponding to, and electrically coupled with each of the plurality of electrode contacts, wherein the plurality of connector pins and the plurality of electrode contacts are covered by the flexible dielectric substrate, a stiffener area affixed to the flexible dielectric material wherein a first side of each of the plurality of electrode contacts are exposed, and a plurality of alignment slots configured to define a separation between each of the plurality of electrode contacts, and a mating connector configured to engage the ECG leadwire connector, wherein the mating connector includes a plurality of alignment tabs and a plurality of mating connector pins, wherein the plurality of alignment tabs engages the plurality of alignment slots of the ECG leadwire connector such that the plurality of electrode contacts maintain an electrical connection with the plurality of mating connector pins.

In another aspect of the present application, an electrocardiograph (ECG) leadwire connector, comprising a plurality of electrode contacts, a plurality of connector pins corresponding to, and electrically coupled with each of the plurality of electrode contacts, a flexible dielectric substrate covering the plurality of connector pins and the plurality of electrode contacts, the flexible dielectric substrate forming the ECG leadwire connecter, a stiffener area formed over the plurality of electrode contacts and affixed to the flexible dielectric material, wherein the stiffener material has a minimum thickness of 0.040 inches, and a plurality of apertures on a first side of the stiffener area and the flexible dielectric substrate wherein the plurality of apertures expose a first side of each of the plurality of electrode contacts, wherein the plurality of aperture have a maximum width of 0.084 inches.

### BRIEF DESCRIPTION OF THE FIGURES

FIG. 1a-1e are graphical representations illustrating test probe calculations in accordance with an embodiment of the present application.

FIG. 2 is a graphical representation illustrating an embodiment of the present application.

FIG. 3 is a graphical representation illustrating an embodiment of the present application.

FIG. 4 is a graphical representation illustrating an embodiment of the present application.

FIG. 5 is a graphical representation illustrating an embodiment of a mating connector of the present application.

FIG. 6a is a graphical representation illustrating an embodiment of a mating connector of the present application.

FIG. 6b is a graphical representation illustrating an embodiment of a mating connector of the present application.

### DETAILED DESCRIPTION

In the present description, certain terms have been used for brevity, clearness and understanding. No unnecessary limitations are to be applied therefrom beyond the requirement of the prior art because such terms are used for descriptive purposes only and are intended to be broadly construed. The different systems and methods described herein may be used alone or in combination with other systems and methods. Various equivalents, alternatives and modifications are possible within the scope of the appended claims. Each limitation in the appended claims is intended to invoke interpretation under 35 U.S.C. §112, sixth paragraph, only if the terms “means for” or “step for” are explicitly recited in the respective limitation.

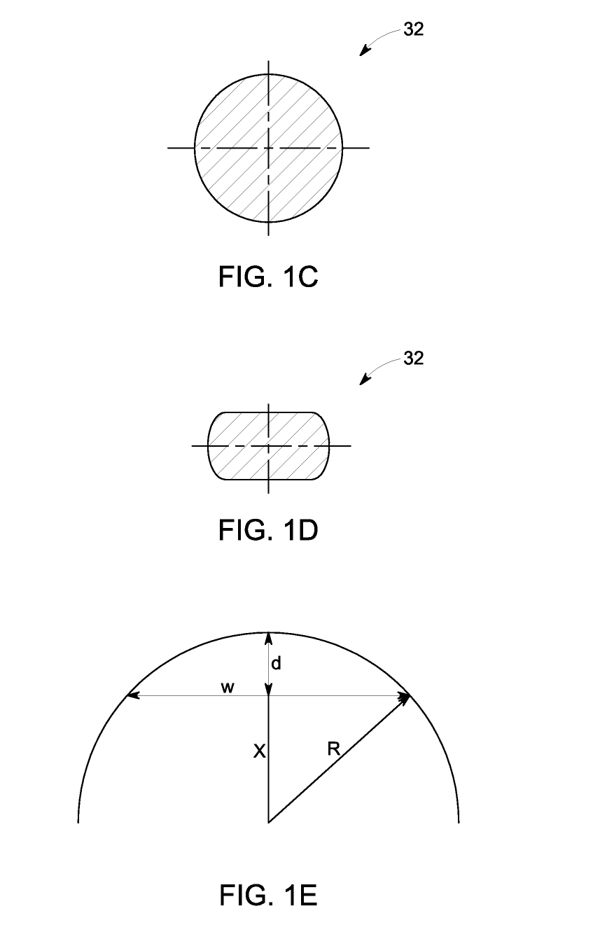

Referring first to FIG. 2, an embodiment includes an ECG leadwire connector 10 design targeted for low cost disposable ECG leadwire applications. This connector 10 implements all of the required patient safety features in a construction that is compatible with the printing and laminating processes used to manufacture disposable ECG leadwires. In addition the connector 10 accommodates multiple leadwire configurations, which are identified by the electrocardiograph (not shown) reading the RFID tag 26 incorporated into the leadwire connector. For example, the RFID tag 26 will be able to identify for the ECG monitor whether the leadwire includes a single lead, a standard 12-lead, or perhaps a 5-lead ECG, or any other ECG leadwire set known in the art. The RFID tag 26 feature is also used to record leadwire usage information and to authenticate the leadwire types being used with the electrocardiograph device. In one embodiment, the RFID tag 26 is fashioned from the flexible printed substrate used to fashion the connector 10 and the ECG leadwires. An RFID chip may then be affixed to the substrate in one embodiment, or readymade RFID tags 26 may be affixed to the leadwire system in the connector 10. Such an RFID tag 26 may be rewritable so that a user knows how long and how many times the particular connector 10 and/or leadwire set has been used. RFID tags are also compatible with the manufacturing process.

Referring now to FIGS. 2-6b, an embodiment includes a disposable ECG leadwire assembly 8 having multiple conductive leadwire traces 19 printed on a flexible dielectric substrate 21. Each conductive trace 19 is protected and isolated from contacting electrical potentials that could be potentially hazardous to the patient along its entire trace 19 length, except at each end. At one end, the conductive trace 19 is connected to an electrode 15 which is intended to be placed at specific locations on the body of the patient where the physiological ECG signal is to be measured. At the electrode contact 16, the conductive trace 19 is exposed to allow an electrical connection to be established, by making contact with a mating connector pin 36 that is part of the mating connector 34. In order to prevent hazardous potentials from inadvertently making contact with the electrode contact 16, a flexible dielectric substrate 21 of prescribed thickness is permanently attached over the ECG leadwire connector 10 of the leadwire assembly 8. This dielectric substrate 18, 21 has small apertures called alignment slots 12, 14 over the areas where the electrode contacts 16 are intended to make contact with the exposed portion of the mating connector pin 36. In addition to these openings, the dielectric substrate 18 material also has narrow slot openings in between the contact access openings in order to introduce an “air gap” between the exposed portion of the connector pins 20. The length of the air gap lot allows the spacing between the exposed portion of the connector pins 20 to be reduced to dimensions that are less than the required “creepage” distance between contacts. Creepage distance is the shortest distance between conductors along any surface. The spacing of the connector pins 20 is then only limited by the required “clearance” distance between contacts. Clearance distance is the shortest distance between conductors not restricted to a path on a material surface. By choosing the proper dielectric substrate 18 thickness, aperture, and slot dimensions, all of the required patient safety requirements can be met when the leadwire connector 10 is either connected to an electrocardiograph or in disconnected states.

These requirements include a specified air “clearance” distance, “creepage” distance, and prevention of contact with the electrode contact 16 when using the “test finger” apparatus specified by the safety regulations on an unmated connector 10. The leadwire connector 10 incorporates an RFID tag 26, which does not require a conductive electrical connection to the electrocardiograph in order for information to be read from or written to the leadwire tag 26. This allows the tag 26 to be embedded into the leadwire connector without impacting the safety or requiring additional leadwire connector pins. The leadwire connector 10 design supports attachment to the electrocardiograph device from several directions, i.e., front or back, and even allows multiple leadwire types to be connected simultaneously in order to perform different types of measurements. However, the connector 10 is configured such that the leadwire assembly 8 may only be connected to an ECG device, and not some other device having a more standard connection point, e.g., network plug, USB. This further reduces the instance of the leadwire assembly 8 being plugged into a dangerous voltage potential that could be hazardous to the patient. It should be further noted that the alignment holes 30, that may be specifically fashioned for a particular ECG device, in other words, as shown in FIG. 2 where there is only one alignment hole 30, are configured such that the connector 10 contacts the correct position in the ECG device such that the alignment slots 12, 14 fit into the ECG device properly, and pin inside the receiver of the ECG device (not shown) receive the alignment hole 30.

Referring now to FIG. 2, an embodiment of the ECG leadwire connector 10 also includes the dielectric coating 18 fashioned around the alignment slots 12, 14, as well as the electrode contact 16 and connector pins 20. The alignment slots 12, 14 provide a contact separation 28 according to the discussion above, and further set forth below. The dielectric coating 18 also provides a trace separation 22 as will further be discussed. The stiffener area 24 allows for an additional material to be fashioned around the dielectric coating 18 such that the connector 10 is rigid enough to be plugged into an ECG device. It should be further noted that there are two different alignment slots 12, 14 wherein one type of slot 12 is longer than the shorter slot 14. The type of slots 12, 14 utilized between any given set of electrode contact 16 may be configured according to the means of the connector 10.

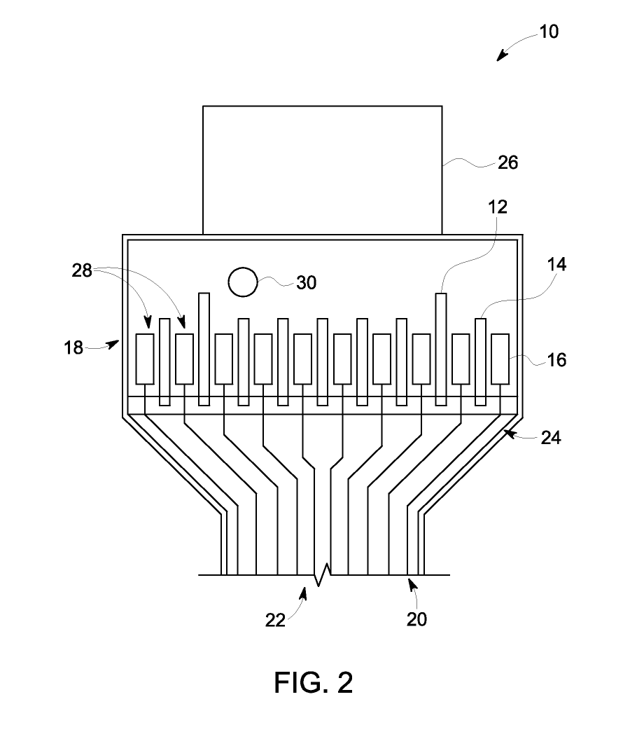

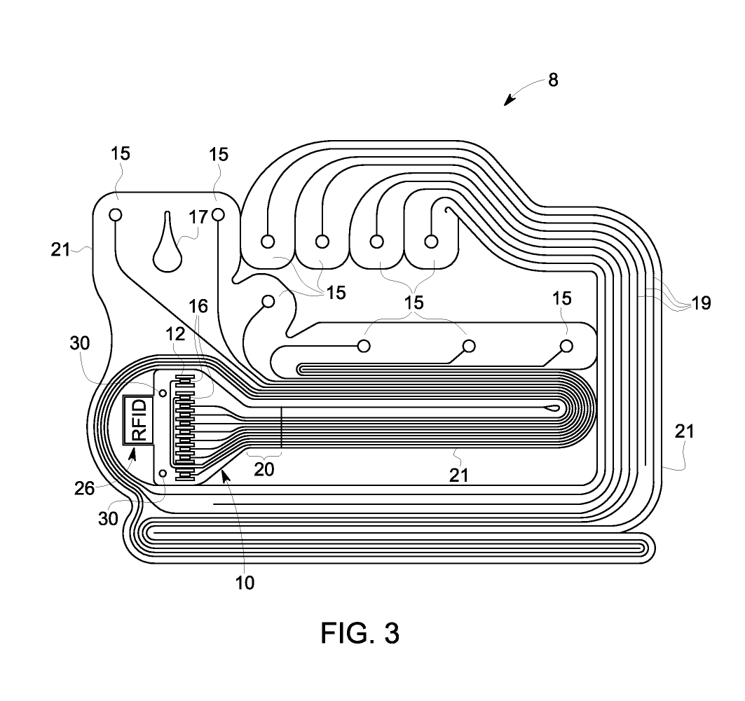

Referring once again to FIGS. 3 and 4, an embodiment of the leadwire assembly 8 also will include an ECG assembly alignment hole 17 in the form of an aperture cut into the flexible dialectric substrate 21 such that the anatomy of the patient may be properly aligned with the ECG assembly alignment hole 17. For example, the ECG assembly alignment hole 17 may be fashioned to be placed on the sternum of a patient with the narrow end of the ECG assembly alignment hole 17 pointing toward the head of the patient.

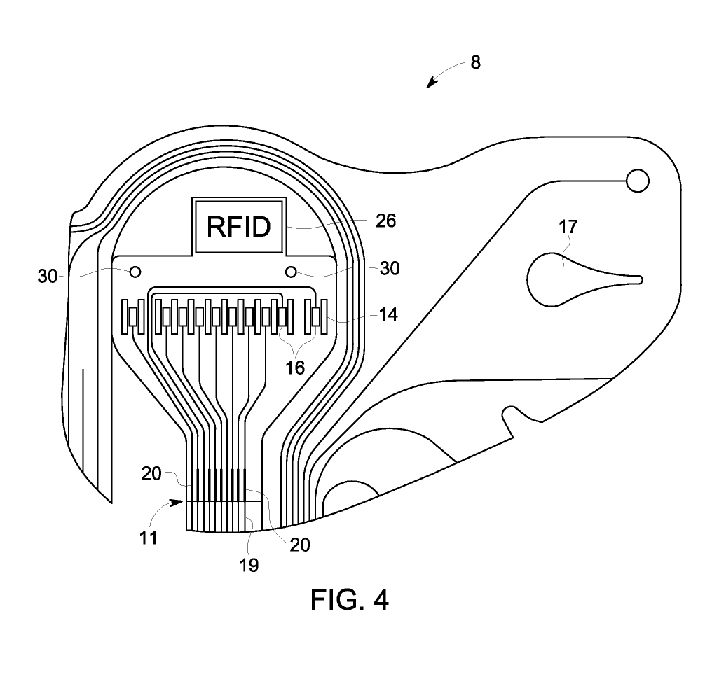

Referring to FIG. 5, an embodiment of a mating connector 34 is illustrated. This mating connector 34 in an embodiment is installed in the ECG monitoring device (not shown), and receives the ECG leadwire connector 10. The mating connector 34 includes alignment tabs 38 that receive the alignment slots 12, 14 of the ECG leadwire connector 10, allowing the electrode contact 16 to maintain an electrical connection with the mating connector pins 36. These pins 36 may then be electrically connected to the appropriate leads of the ECG monitoring device.

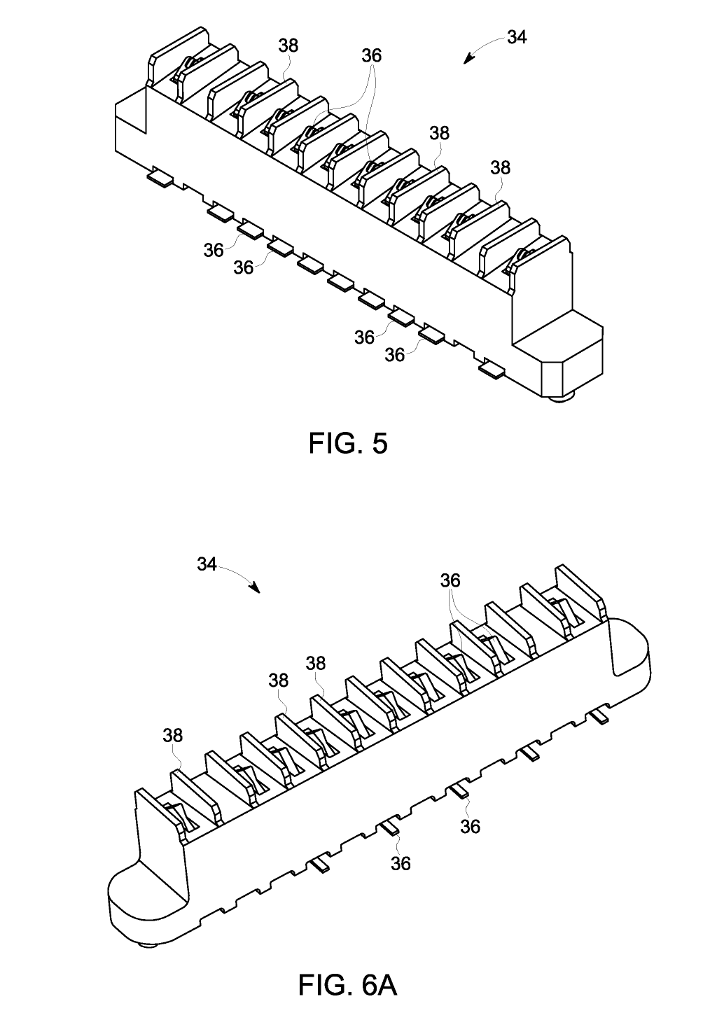

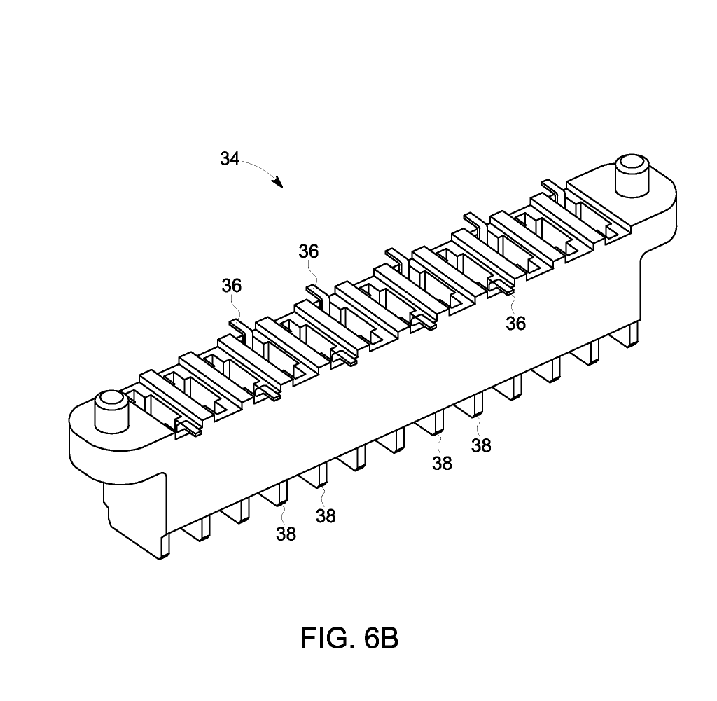

Referring to FIGS. 6a and 6b, a further embodiment of the mating connector 34 is illustrated. Once again, this mating connector 34 includes a number of alignment tabs 38 for receiving alignment slots 12, 14 and mating connector pins 36. In FIG. 6b, the mating connector 34 is inverted in order to illustrate the configuration of the mating connector pins 36.

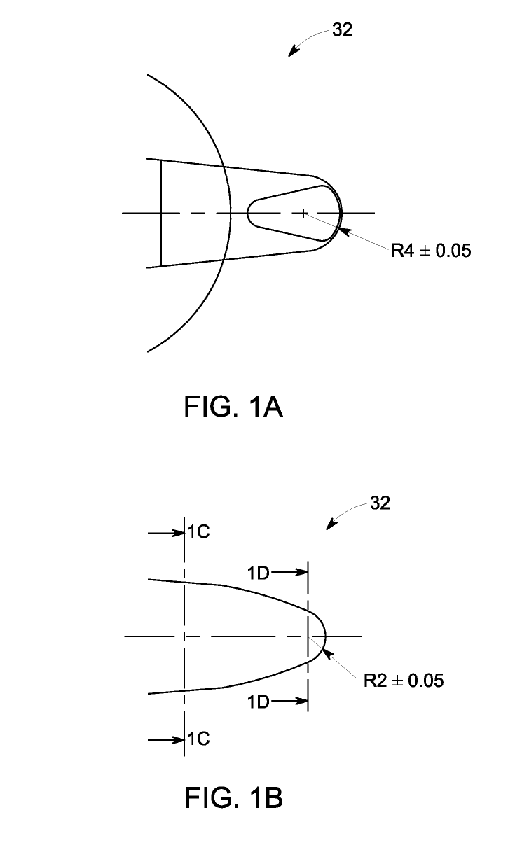

Referring now to FIGS. 1a-e, these graphical representations illustrate test probe calculations according an embodiment. The tip of the test probe 32 has two radiuses as shown in FIGS. 1a and 1b. In FIG. 1a, the radius R4 is 4.0 mm+/−0.05 mm, and in FIG. 1b, the radius R2 is 2.0 mm+/−0.05 mm. The larger of the two radii R4 would prevent the test probe 32 from completely entering an opening that is larger than 2.0 mm but smaller than 4.0 mm. FIGS. 1c and 1d illustrate cross-section IC and ID of FIG. 1b. To simplify the worst case analysis below, only the smaller radii R2 is considered. If the requirements are met under this simplified analysis, inclusion of the effects of the larger radii R4 will only improve the margin by which they are met.

| | | |
|---|---|---|
| Smallest probe tip radius = | 1.95 mm | 0.077 in |

Referring to FIGS. 1a-1e and FIG. 2 simultaneously, the leadwire connector 10 dimensions with respect to making electrical contact with the contacts 16 below the stiffener area 24 is the thickness of the stiffener material, which is 0.045 in+/−0.005 in, and the width of electrode contact 16 opening, which is 0.0785 in+/−0.005 in. Of course, further embodiments could be designed such that the opening size is based on the stiffener thickness to inhibit the finger probe from getting closer than a prediscribed distance from a protected conductive surface.

| | | |
|---|---|---|
| minimum stiffener thickness = | 0.040 in | 1.016 mm |
| maximum contact opening = | 0.084 in | 2.1209 mm |

FIG. 1c represents the geometry of the penetration distance d of the test probe 32 tip with radius R, into the stiffener opening slot width w, as calculated using the following equations (1)-(3):

$$\begin{matrix}X = \sqrt{{R}^{2} - \frac{{w}^{2}}{4}} & ( 1 ) \\ d = R - X & ( 2 ) \\ d = R - \sqrt{{R}^{2} - \frac{{w}^{2}}{4}} & ( 3 )\end{matrix}$$

where d represents the penetration depth of the test probe 32 into the connector 10. The air clearance distance would be approximately the stiffener thickness minus the penetration depth and the creepage distance would be at least that much and likely be equal to the stiffener thickness itself.

Therefore, the ECG leadwire connector 10 worst case test probe 32 penetration depth and air clearance distance is:

| | | |
|---|---|---|
| d = | 0.012 in | 0.314 mm |
| worst case air clearance = | 0.028 in | 0.702 mm |
| creepage distance | 0.040 in | 1.016 mm |

Based on this analysis, the test probe 32 tip cannot make contact with the electrode contact 16 within the connector 10, and the worst case creepage distance is greater than the minimum 1.0 mm specified by the standards, excluding stiffener material compression affects. This analysis covers the 1,500 volt standards requirements which are intended to protect the patient from hazards associated with inadvertent contact with power mains or other hazardous potentials. A secondary aspect of the design which attains the 5,000V isolation associated with the maximum of 10% reduction in energy delivered by a defibrillator is accounted for by the 2 times the stiffener thickness plus the spacing between the contacts (i.e., air clearance, 4.0 mm minimum), and 2 times the stiffener thickness plus the surface distance around the ends of the air gap slots (i.e. creepage, 8.0 mm minimum).

The connector 10 stiffener area 24 is made from a semi-rigid material which could have some deformation when subjected to a 30 N (6.744 lbs.) force, so the impact of this test probe 32 force on the creepage and air clearance distances may not be negligible. Materials for the stiffener area 24 may be selected from any appropriate materials which do not deform substantially under the indicated test finger probe pressure.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

I claim:

1. An electrocardiograph (ECG) leadwire connector, comprising:
a plurality of electrode contacts;
a plurality of connector pins corresponding to, and electrically coupled with each of the plurality of electrode contacts, wherein the plurality of connector pins and the plurality of electrode contacts are covered by a flexible dielectric substrate, the flexible dielectric substrate forming the ECG leadwire connecter;
a stiffener area formed over the plurality of electrode contacts and affixed to the flexible dielectric material;
a plurality of apertures on a first side of the stiffener area and the flexible dielectric substrate wherein the plurality of apertures expose a first side of each of the plurality of electrode contacts; and
a plurality of alignment slots configured through the stiffener area and the flexible dielectric material, wherein the plurality of alignments slots define a separation between each of the plurality of electrode contacts.

2. The ECG leadwire connector of claim 1, further including a radio frequency identification (RFID) tag, wherein the RFID tag is affixed to the ECG leadwire connector such that the ECG leadwire connector may be identified by an ECG monitoring device.

3. The ECG leadwire connector of claim 1, wherein the ECG leadwire connector engages with a mating connector in the ECG monitoring device.

4. The ECG leadwire connector of claim 3, wherein the mating connector includes a plurality of alignment tabs and a plurality of mating connector pins, wherein the plurality of alignment tabs engages the plurality of alignment slots of the ECG leadwire connector such that the plurality of electrode contacts maintain an electrical connection with the plurality of mating connector pins.

5. The ECG leadwire connector of claim 1, further including at least one alignment hole, wherein the at least one alignment hole engages the ECG monitoring device such that the ECG leadwire connector engages the ECG monitoring device in a proper position.

6. The ECG leadwire connector of claim 1, wherein the plurality of alignment slots maintain an electrode contact separation distance of at least a required creepage distance.

7. The ECG leadwire connector of claim 1, wherein the flexible dielectric substrate maintains a separation between each of the plurality of connector pins that is at least a required clearance distance.

8. The ECG leadwire connector of claim 1, wherein each of the connector pins of the ECG leadwire connector are electrically coupled and physically coupled to a leadwire assembly.

9. The ECG leadwire connector of claim 8, further including a plurality of conductive traces in the leadwire assembly, wherein the conductive traces are electrically coupled the plurality of connector pins and further coupled to a plurality of ECG electrodes.

10. An electrocardiograph (ECG) leadwire system, comprising:
an ECG leadwire connector fashioned from a flexible dielectric substrate having a plurality of electrode contacts and a plurality of connector pins corresponding to, and electrically coupled with each of the plurality of electrode contacts, wherein the plurality of connector pins and the plurality of electrode contacts are covered by the flexible dielectric substrate, a stiffener area affixed to the flexible dielectric material wherein a first side of each of the plurality of electrode contacts are exposed, and a plurality of alignment slots configured to define a separation between each of the plurality of electrode contacts; and
a mating connector configured to engage the ECG leadwire connector, wherein the mating connector includes a plurality of alignment tabs and a plurality of mating connector pins, wherein the plurality of alignment tabs engages the plurality of alignment slots of the ECG leadwire connector such that the plurality of electrode contacts maintain an electrical connection with the plurality of mating connector pins.

11. The ECG leadwire system of claim 10, further including a radio frequency identification (RFID) tag, wherein the RFID tag is affixed to the ECG leadwire connector such that the ECG leadwire connector may be identified by an ECG monitoring device.

12. The ECG leadwire system of claim 10, further including at least one alignment hole, wherein the at least one alignment hole engages the ECG monitoring device such that the ECG leadwire connector engages the ECG monitoring device in a proper position.

13. The ECG leadwire system of claim 10, wherein the plurality of alignment slots maintain an electrode contact separation distance of at least a required creepage distance.

14. The ECG leadwire system of claim 10, wherein the flexible dielectric substrate maintains a separation between each of the plurality of connector pins that is at least a required clearance distance.

15. The ECG leadwire system of claim 10, wherein each of the connector pins of the ECG leadwire connector are electrically coupled and physically coupled to a leadwire assembly.

16. The ECG leadwire system of claim 15, further including a plurality of conductive traces in the leadwire assembly, wherein the conductive traces are electrically coupled the plurality of connector pins and further coupled to a plurality of electrodes.

17. An electrocardiograph (ECG) leadwire connector, comprising:
a plurality of electrode contacts;
a plurality of connector pins corresponding to, and electrically coupled with each of the plurality of electrode contacts;
a flexible dielectric substrate covering the plurality of connector pins and the plurality of electrode contacts, the flexible dielectric substrate forming the ECG leadwire connecter;
a stiffener area formed over the plurality of electrode contacts and affixed to the flexible dielectric material, wherein the stiffener material has a minimum thickness of 0.040 inches; and
a plurality of apertures on a first side of the stiffener area and the flexible dielectric substrate wherein the plurality of apertures expose a first side of each of the plurality of electrode contacts, wherein the plurality of aperture have a maximum width of 0.084 inches.
